(19) United States
(12) Patent Application Publication
(10) Pub. No.: US 2021/0204858 A1
(43) Pub. Date: Jul. 8, 2021

(54) AUTOMATIC SENSING OF FEATURES WITHIN AN ELECTROCARDIOGRAM

(71) Applicant: Mayo Foundation for Medical Education and Research, Rochester, MN (US)

(72) Inventors: Itzhak Zachi Attia, Rochester, MN (US); Paul A. Friedman, Rochester, MN (US); Peter A. Noseworthy, Rochester, MN (US)

(21) Appl. No.: 17/058,313

(22) PCT Filed: May 23, 2019
(86) PCT No.: PCT/US2019/033678

§ 371 Date: Nov. 24, 2020

Related U.S. Application Data
(60) Provisional application No. 62/678,803, filed on May 31, 2018.

(51) Int. Cl. A61B 5/358; A61B 5/00; A61B 5/349; A61B 5/355; G16H 50/20; G06N 20/00; G06N 5/00

(52) U.S. Cl. CPC A61B 5/358; A61B 5/7267; A61B 5/349; G06N 5/003; G16H 50/20; G06N 20/00; A61B 5/355

(57) ABSTRACT

Data is generated that describes features of an ECG of a subject. This generation can include receiving ECG data that was generated to reflect cardiac activity of a particular mammal; submitting the ECG data to a plurality of cardiac classifiers, each cardiac classifier configured to identify, in the ECG, at least some of a plurality of cardiac features that are within a particular feature-class; receiving, from each of the plurality of cardiac classifiers, a classification message containing data of the cardiac classifiers identifying of cardiac features in the ECG; and assembling, from the received classification messages, ECG features for the ECG, the ECG features identifying at least some features of different feature-classes.

### CROSS-REFERENCE TO RELATED APPLICATIONS

This application claims priority to U.S. Application Ser. No. 62/678,803, filed on May 31, 2018. The disclosure of the prior application is considered part of the disclosure of this application, and is incorporated in its entirety into this application.

### TECHNICAL FIELD

This document describes computer-based technology for automatic processing of electrocardiogram data.

### BACKGROUND

Electrocardiography (ECG or EKG) can include the process of recording the electrical activity of the heart over a period of time using, for example, electrodes placed on the skin. These electrodes detect the tiny electrical changes on the skin that arise from the heart muscle's electrophysiologic pattern of depolarizing and repolarizing during each heartbeat.

### SUMMARY

This specification discloses systems, methods, devices, and other techniques for identifying features of an electrocardiogram (ECG) of a subject, including at least one sensor contactable with the subject; and an element adapted to receive signals from said sensor and configured to determine a plurality of features of an ECG generated from the signals by submission of the ECG to a plurality of machine-learning classifiers that are each configured identify some, but not all, features out of a complete feature-set. [5] This specification discloses systems, methods, devices, and other techniques for generating data that describes features of an ECG of a subject including one or more hardware processors; and non-transitory computer-readable memory containing instructions that, when processed by the processor, cause the system to perform operations including receiving ECG data that was generated to reflect cardiac activity of a particular mammal; submit the ECG data to a plurality of cardiac classifiers, each cardiac classifier configured to identify, in the ECG, at least some of a plurality of cardiac features that are within a particular feature-class; receive, from each of the plurality of cardiac classifiers, a classification message containing data of the cardiac classifiers identifying of cardiac features in the ECG; assembling, from the received classification messages, ECG features for the ECG, the ECG features identifying at least some features of different feature-classes.

Implementations can include some, all, or none of the following features. The plurality of cardiac classifiers include a main-rhythm-classifier configured to identify, in the ECG, at least some main-rhythm features; a secondary-rhythm-classifier configured to identify, in the ECG, at least some secondary-rhythm features; an atrial-enlargement-classifier configured to identify, in the ECG, at least some atrial-enlargement features; an atrioventricular (AV)-conduction main-rhythm-classifier configured to identify, in the ECG, at least some AV-conduction features; a QRS-classifier configured to identify, in the ECG, at least some QRS-features; a ST\T-wave-classifier configured to identify, in the ECG, at least some ST\T-wave features; a noisy-classifier configured to identify, in the ECG, at least some noisy features. At least some of the cardiac classifiers are configured to access physiological-constraint data that defines constraints on possible feature identification such that identified features are constrained to only features that are physiologically possible in a single given ECG. At least one of the cardiac classifiers are configured to identify three or more cardiac features that are within a particular feature-class. A second plurality of cardiac classifiers are at least some of the cardiac classifiers that are each configured to identify cardiac features within a particular feature-class. The second plurality of cardiac classifiers are arranged in a decision tree such that identification of some cardiac features by a first cardiac classifier of the second plurality of cardiac classifiers causes a second cardiac classifier to identify at least one cardiac feature within the particular feature-class.

Implementations of the technology described in this document can be used to realize all, some, or none of the following advantages. Technology used in the recognition of features of an ECG is improved. ECGs can be recognized in an automated fashion, requiring little or no particular user input or feedback to classify an ECG. In some cases, immediate and/or preliminary ECG identification can be provided to a skilled ECG reader (e.g., a cardiologist) to assist the ECG reader in understanding the ECG. In some cases, automated ECG identifications can be provided to an unskilled ECG reader (e.g., an end user or health-care provider that is not exert at ECG analysis). In some cases, each neural network is trained to classify only a subset of all the features (or cardiac conditions, they can be trained on less data, and can be trained on a more balanced distribution of data. Some features/conditions may be exhibited in relatively few training samples, while others are quite common (e.g., normal sinus rhythm). By using these techniques, an otherwise insufficient quantity of training samples for the rare condition may be used to generate more-accurate classifiers. With greater degree of parity in the sample sizes among the features, classifier that classifies only a subset of all possible features/cardiac conditions.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a conceptual diagram of an example system for recording and processing ECG data to identify features of the ECG.

FIG. 2 is a block diagram of example data that can be used in the processing of ECG data.

FIG. 3 is a block diagram of an example system for processing ECG data.

FIG. 4A is a flowchart of an example process for processing ECG data.

FIG. 4B is a flowchart of an example process for processing ECG data.

FIG. 5 is a block diagram of example data that can be used in the processing of ECG data.

FIG. 6A-6C is a spreadsheet of example data that can be used in the processing of ECG data.

FIG. 7 is a schematic diagram that shows an example of a computing system.

FIG. 8 show a graph of measured cardiac action.

Like references and indicators among the drawings indicate like elements.

### DETAILED DESCRIPTION

This document describes technology used for the automatic identification of features of an electrocardiogram (ECG) of a mammal. In some cases, this can involve the submission of an ECG to a classification engine that includes components that each are configured to identify some of the features of the ECG to the exclusion of other features. In particular, machine-learning classifier sets may be trained to identify one or more features of a particular class of features within the ECG. Then, these features from each classifier set may be combined to generate a complete list of features found in the ECG.

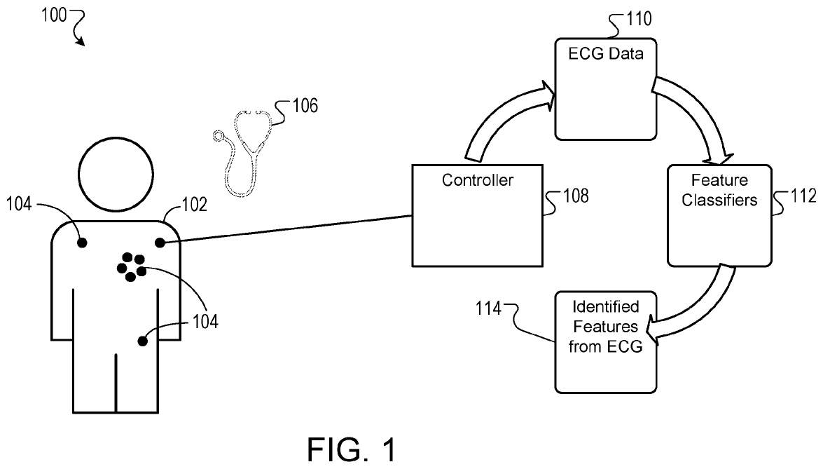

FIG. 1 is a conceptual diagram of an example system 100 for recording and processing ECG data 110 to identify features of the ECG. For the purpose of this example, the subject 102 will be considered as a human, and more specifically as a patient of a healthcare provider. However, it should be understood that the description is not limited to this example. In other implementations, the subject 102 may be a human who is not specifically associated with a healthcare provider, or may be any other mammal for which an appropriate model has been constructed to identify features of ECG data 110.

A set of electrodes 104 are disposed on a surface of the patient 102 to enable recording of signals indicating the cardiac electrical activity of the patient 102 during an ECG procedure. In some implementations, 10 leads may be affixed to a patient to perform a standard 12-lead ECG recording (e.g., with several electrodes located on the chest near the region of the heart and other electrodes located on the limbs of the patient). The 12-lead ECG is beneficial to obtain multiple channels of ECG data 110, where the data for each channel represents a respective lead. Each lead is formed by the electrical potential between a pair of electrodes. Due to variances in electrode positioning, each lead provides a different view of the patient's cardiac electrical activity as a result of the different angles formed by the different pairs of electrodes for the different leads. For example, the signal from each lead can be recorded simultaneously for a period of time (e.g., 5, 10, or 15 seconds) to capture information about the timing and location of electrical activity along different radial directions.

In some implementations, ECG data 110 can be recorded using a sensor platform or electrode configuration other than, or in addition to, the 12-lead ECG configuration. For example, a removable ECG patch may be affixed to the surface of a patient, where the patch includes two or more electrodes forming one, two, or more leads from which an ECG can be recorded. In some implementations, the patient 102 can manually contact a fixed pair of external electrodes with his or her fingers, or the patient 102 may wear a watch, wristband, chest band, or other device that secures two or more electrodes in position on the patient 102 to sense the patient's cardiac electrical activity. A mobile computing device (e.g., smartphone) may be used by the patient 102 or a healthcare provider to configure aspects of the ECG procedure in a clinical or non-clinical setting. A stethoscope 106 may be positioned on the patient 102 to sense the patient's cardiac electrical activity. In some implementations, other elements of the system 100 such as the controller 100 may be incorporated into the electrodes 104, mobile computing device, stethoscope 106, etc. In some implementations, other elements of the system 100 may be mechanically separated and in data communication with the electrodes 104, mobile computing device, stethoscope 106, etc.

A controller 108 can receive the ECG data 110 and organize the ECG data 110 into a useable format. For example, the controller 110 can include a pre-processor that digitally conditions ECG signals received by the controller 108. The pre-processor may perform noise reduction, anti-aliasing, or other digital techniques to prepare ECG data 110 describing the ECG of the patient 102 for further processing.

This further processing can include submitting the ECG data 110 to one or more feature classifiers 112. As will be described in further detail below, when ECG data 110 is submitted to the feature classifiers 112, the feature classifiers 112 identify one or more identified features 114 within the ECG data 110. For example, a feature classifier may be configured to identify if an ECG contains sinus rhythm (or not), what kind of sinus rhythm, AV-conduction abnormalities (or not), arrhythmia (or not), etc. In some cases, these feature classifiers may be generated via machine learning. That is, using a very large dataset of ECG data known to have various features, machine learning techniques can examine this dataset and construct feature classifiers that are able to determine if a new ECG data 110 is likely to or not likely to have some feature. This identification may take the form of, for example, a confidence level (e.g., 80% confidence that Normal Sinus Rhythm is present), a list of most likely features (e.g., with sinus arrhythmia or with marked sinus arrhythmia), or a single feature or set of features (e.g., sinus rhythm with atrial complexes). In some implementations, some or all of the feature classifiers may be generated using non-machine learning techniques. This may include a mix of machine-learning feature classifiers and non-machine-learning classifiers.

As shown in FIG. 1, the identified features from ECG may be generated without device input from a trained cardiologist. For example, as part of patient intake, in an emergency situation, in a home environment, or in a remote environment, an unskilled user or non-cardiologist may use the system 100 to screen the patient 102 for cardiac health. In one example, a patient may visit a general practitioner's office for a yearly physical. As part of collecting the patient's vital statistics, a nurse practitioner may use the stethoscope 106 to generate the ECG data and thus the identified features 114. Any abnormal features in the identified features 114 can be flagged by the stethoscope for further investigation by the nurse practitioner, a non-cardiac medical doctor such as the general practitioner, or for referral to a specialized cardiologist.

Although not shown in FIG. 1, the system 100 may be used by a trained and specialized cardiologist to assist in the identification of features in ECG data including the ECG data 110. For example, a cardiologist may be tasked with identifying features in many (e.g., dozens or hundreds) of ECGs. In order to more efficiently do so, the cardiologist may be presented with the identified features 114 as proposed or preliminary features that the cardiologist can either accept or override based on their specialized training. In this way, the cardiologist can be enabled to more quickly and efficiently process ECG data.

In one example, the cardiologist may have a queue of ECG readings to process. This queue of ECG data can be automatically analyzed to generate identified features in each ECG reading, and the queue can be sorted based on a risk metric from those identified features. That is, ECG readings that are automatically identified to potentially contain life-threatening features can be sorted to the front of the queue, while ECG readings without any unhealthy features automatically identified can be sorted toward the back of the queue. In this way, the queue of awaiting ECG readings can be automatically triaged for the cardiologist so that the most critical ECG readings can be handled by the cardiologist soonest.

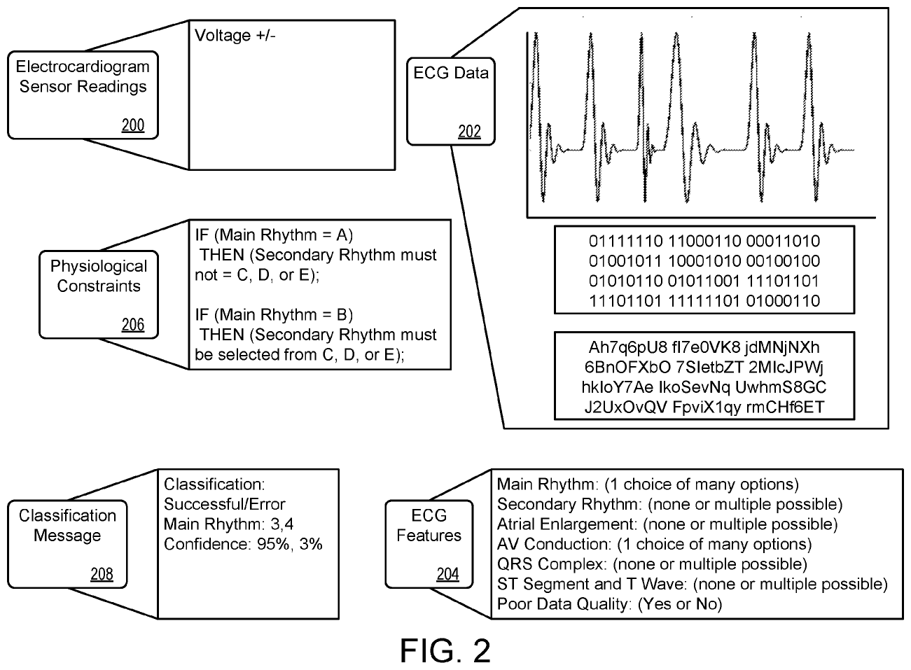

FIG. 2 is a block diagram of example data that can be used in the processing of ECG data.

Electrocardiogram sensor readings 200 include data or signals sent, for example, from the electrodes 104 to the controller 108. The electrocardiogram sensor readings 200 can include, for example, one or more voltage readings that reflect a voltage difference between two points on the body of the patient 102.

ECG data 202 includes computer-readable data stored or handled by a computer that reflects cardiac action of the patient 102. The ECG data 202 can be rendered into a visual representation that shows activity (e.g., electrical activity such as depolarization and repolarization) over time. The ECG data 202 can be stored in computer-readable formats such as a string of binary (e.g., 1 and 0) characters or other computer readable characters such as ASCII or hexadecimal characters. Depending on the configuration of the system using the ECG data 202, the ECG data 202 may take the format of one or more well-known or private data formats such as, but not limited to, SCP-ECG, DICOM-ECG, and HL7 aECG.

ECG features 204 include data identifying one or more features that have been identified in an ECG (i.e., ECG data 202.) In some cases, ECG features 204 may include all features that have been identified within ECG data 202. In some cases, ECG features 204 may include only a subset of all features identified within ECG data 202. For example, a feature classifier may only identify QRS Complex features, and thus ECG features 204 generated by that feature classifier may only include information about those type of features.

The ECG features 204 may be formatted as a structured data type. For example, the ECG features 204 may be organized into field, with each fields holding one or more values. Each of these fields may be related, for example, to one class of features that can be identified by feature classifiers. In one scheme, these fields include main rhythm, secondary rhythm, atrial enlargement, AV-conduction, QRS complex, ST Segment and T wave, and poor data quality (to store data indicating if the associated ECG data 202 is of poor quality due to noise, etc.) This data may be stored as, for example, an extensible markup language (XML) object, a JavaScript Object Notation (JSON), an entry in a database, or any other appropriate computer-readable format.

Physiological constraints 206 include data that explicitly identifies constraints on possible values within a set of ECG features 204.

In some implementations, physiological constraints are implicitly defined. For example, the structure of feature identifiers may be such that physiologically-impossible combinations of features may never be produced. The physiological constraints may be used, for example, by feature classifiers in order to ensure that two different classifiers do not independently produce results that would incompatible with each other. For example, one feature classifier may determine that an ECG data is likely to contain “sinus bradycardia” and another feature classifier may determine that the same ECG data is likely to contain “sinus tachycardia.” Given that this is a physiologically impossible combination, a system may determine that the physiological constraints 206 prevents such a combination and the system may avoid combining these two features into a single set of features to be presented to a user. For example, the feature set may offer these to features as an “either/or” combination (i.e. either normal sinus rhythm” or “sinus arrhythmia), possibly with confidence rating for each. In some examples, the set of these two features may be set aside and sets of features that are not constrained out by the physiological constraints 206 may be provided to a user.

Classification messages 208 include messages generated by feature classifier to communicate the feature classifier's results. For example, after a feature classifier successfully identifies a feature (including successfully identifying no such features if they are not present), the feature classifier may pass a classification message 208 back to a controller and/or another feature classifier.

In this example, the classification message 208 can include a field to specify if the classification completed successfully or failed, a listing of one or more identified features, and confidence value for each such identified features. However, more, less, and/or different data could be included. This data may be stored as, for example, an extensible markup language (XML) object, a JavaScript Object Notation (JSON), an entry in a database, or any other appropriate computer-readable format.

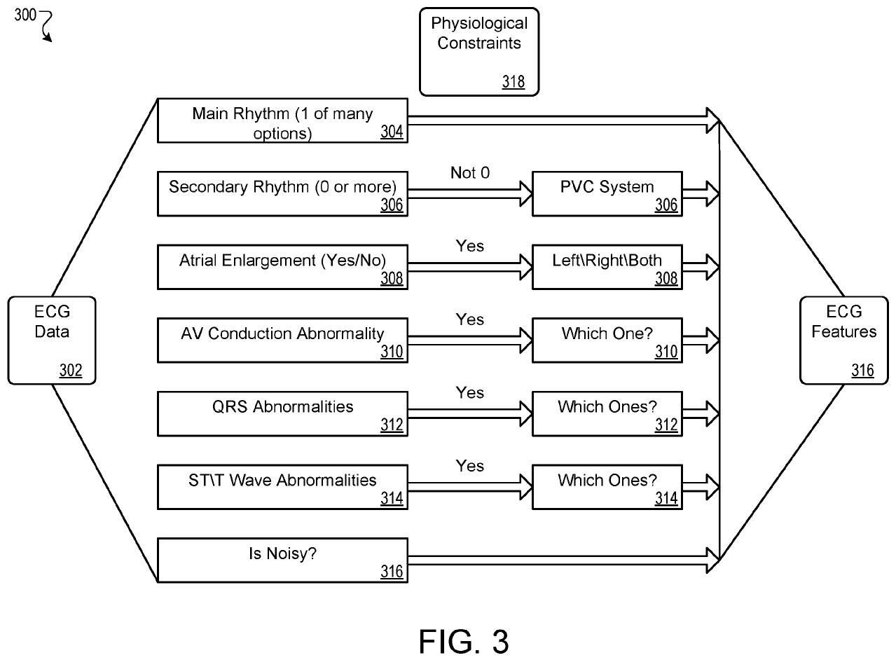

FIG. 3 is a block diagram of an example system 300 for processing ECG data 302. For example, the system 300 can be used by the controller 108 in order to generate the identified features 114 from the ECG data 110. Elements of the system 100 may be implemented as, for example, one or more applications executing on a computing device (e.g., desktop computer or a mobile computing device like a phone), a medical device (e.g., a stethoscope or ECG device), or a cloud-computing environment (e.g., on one or more physical or virtual servers).

In order to process the ECG data 302 and generate the ECG features 316, the system 300 can pass the ECG data 302 to feature-groups 304-316. Each feature-group 304-316 can include one or more feature classifiers that are all configured to identify cardiac features that are within a particular feature-class. For example, for the feature-class “QRS Abnormalities,” the feature-group 312 can include one or more feature classifiers configured to identify QRS abnormalities, including the absence of such abnormalities. In addition to the feature classifiers, each of the feature-groups 304-316 can include other elements that support the function of the feature-groups 304-316. For example, message passing elements, data storage and access elements, etc. may also be included in the feature-groups 304-316. Messages passed between the feature-groups 304-316 and with other elements of the system 300 can include classification messages like the classification messages 208 previously described.

In some implementations, the feature-groups 304-316 can be run in parallel and concurrently. For example, in some configurations, each of the feature-groups 304-316 may be independent of the other feature-groups 304-316. In such a case, each feature-groups 304-316 may be passed a copy of the ECG data and possibly other input data such as the physiological constraints 318, with none of the feature-groups 304-316 requiring the output of another feature group 304-316 as input. In some implementations, one or more feature-groups 304-316 may require as input the output of another feature-group 304-316.

Features identified by each of the feature groups 304-316 may be aggregated into a final collection of features in the ECG features 316. For example, the system 300 may collect classification messages 208 from the feature groups 304-316 and generate ECG features 316 from the feature messages. The system 300 can respect the constraints of the physiological constraints 318 when assembling the ECG features 316 to ensure that only physiologically valid feature combinations are generated.

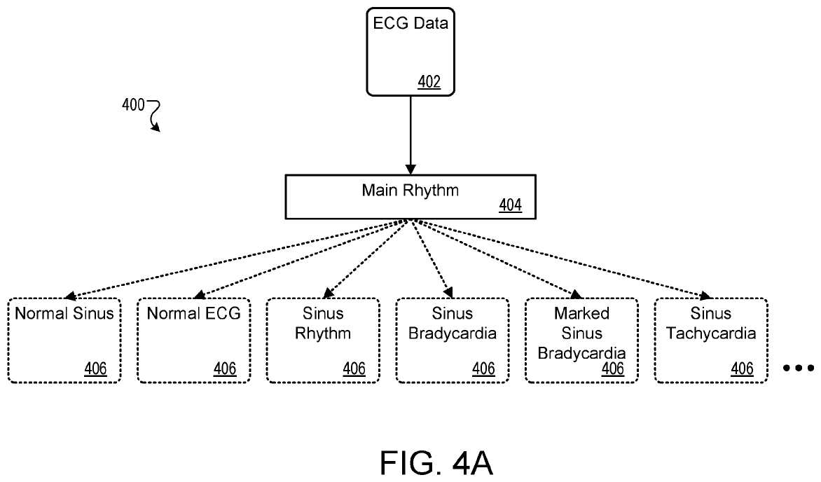

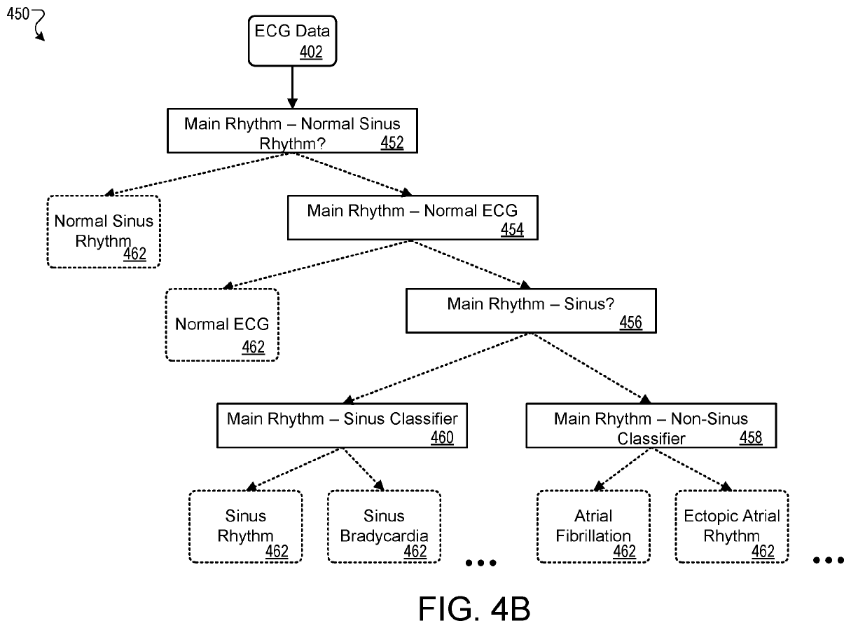

FIG. 4A is a flowchart of an example process 400 for processing ECG data. The process 400 can be used by, for example, a feature-group tasked with identifying the main-rhythm features of an ECG (e.g., feature-group 304). In this example, the feature-group has only one feature classifier, a single main-rhythm classifier.

The main-rhythm classifier receives ECG data 402 for processing (404). For example, the main-rhythm classifier is passed the ECG data 402 by a system that also passes the ECG data 402 to other feature-groups that are each tasked with identifying non-main-rhythm features in the ECG data 402.

The main-rhythm classifier has been trained on a large corpus of tagged ECG data using machine-learning techniques in order to identify main-rhythm features in new ECG data. This training may include, for example, the use of linear classifiers (e.g., Fisher's linear discriminates, logistic regression, Naive Bayes classifiers, perceptrons), support vector machines (e.g., least square support vector machines), quadratic classifiers, kernel estimation (e.g., k-nearest neighbor engines), decision trees (e.g., random forests), neural networks, learning vector quantization, or any other appropriate machine-learning process. In some cases, the main-rhythm classifier (or any other feature classifier) may include or instead be non-machine learning classifiers. For example, human-crafted heuristic models may be used.

In some configurations, the main-rhythm classifier may operate as a function. As input, the main-rhythm classifier may use the ECG data 402. As output, the main-rhythm classifier may generate a classification message that includes one or more main-rhythm tags 406. These tags may be all features identified, all features identified as having a confidence score greater than some threshold N, the Mtop features, etc. Additionally, the classifier may contain other data as required by the system.

FIG. 4B is a flowchart of an example process 450 for processing ECG data 402. Unlike in the process 400, the process 450 uses a plurality of feature classifiers arranged in a decision tree in order to generate a classification message with one or more main-rhythm tags 406. In the process 450, the feature classifiers are each configured to identify cardiac features within a particular feature-class (i.e. main-rhythm features). Further, the feature classifiers are arranged in a decision tree such that identification of some cardiac features by a first cardiac classifier of the second plurality of cardiac classifiers causes a second cardiac classifier to identify at least one cardiac feature within the particular feature-class. Therefore, depending on the outcome of any particular feature classifier, the process 450 may terminate with the generation of a classification message, or may continue on to the next element of the process 450.

One or more main-rhythm classifier receives the ECG data 402 for processing (452-460). For example, the main-rhythm classifiers may be arranged into a decision tree. The ECG data 402 may be passed to the head of the tree (452) for initial processing. The head of the tree may be a main-rhythm classifier configured to determine if the ECG data 402 contains normal sinus rhythm or not. If the main-rhythm classifier determines that the ECG data 402 does (or likely does) contain normal sinus rhythm, a classification message containing a tag 462 indicating as such can be generated. If the main-rhythm classifier determines that the ECG data 402 does not (or likely does not) contain normal sinus rhythm, a normal ECG classifier can receive the ECG data 402 for processing (454). The process 450 can continue through the decision tree until a leaf is reached, at which point a classification message containing one or more tags 462 is generated. [47] As shown, the process 450 uses a decision tree that is not binary. That is, at least one node (at 458 and 460) contain more than two branches, as indicated by the ellipses. However, other processes may use a strict binary tree where each node contains two and only two branches.

The process 400 and the process 450 demonstrate two different way that feature classifiers may be arranged within a single feature-group. That is to say, a feature group may include one feature classifier, or the feature group may include many feature classifiers. For example, the feature-group 304 may be configured to perform the operation 400, or the feature-group 304 may be configured to perform the operation 450. Similarly, another feature-group may be configured to perform operations that include only one feature classifier, or may be configured to perform operations that include many feature classifiers.

Use of a decision tree as is done in the process 450 may improve the technology of feature identification in ECG data. For example, many features in a particular feature class may be very rare compared to the most common features. Use of a decision tree can allow for the use of classifiers that have only been trained to discriminate between two (or a small number of) rare features without concern for the much more common features. This can allow these special-case classifiers to be made much more accurate in cases in which common features have been ruled out. Additionally, this can advantageously allow for the creation of classifiers on smaller corpuses of the rare features.

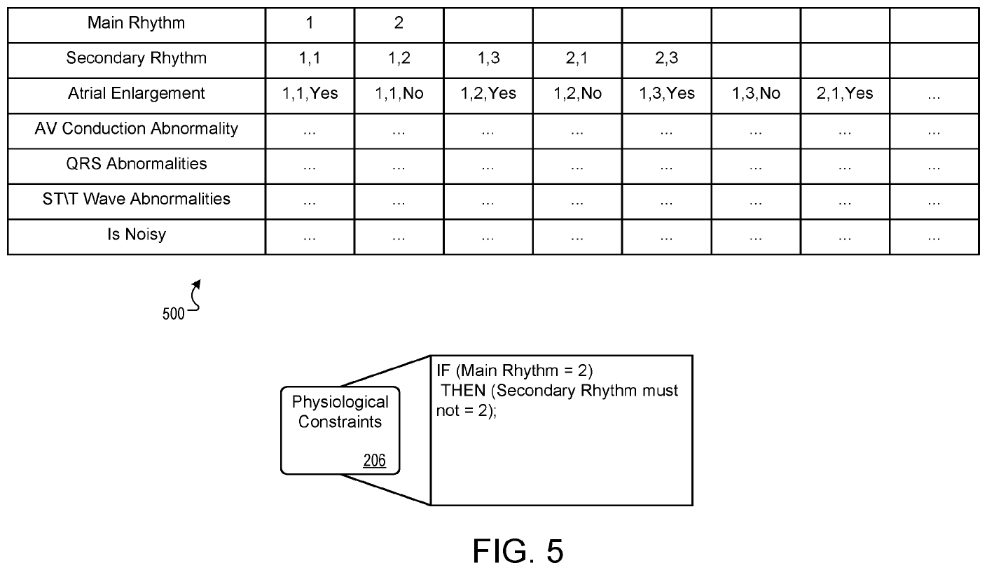

FIG. 5 is a block diagram of example data 500 that can be used in the processing of ECG data. In this example, the data 500 conforms to the physiological constraints 206. Each row of the data 500 represents possible ECG features (e.g., ECG features 204). For clarity, with each successive row representing additional features added by various feature groups (e.g., feature-groups 304-316). In this example, the rows are created in the same order the feature-groups 304-316 are shown in, but other orders are possible. Indeed, in many systems when feature-groups 304-316 are processed in parallel, different orders would be likely and a product of complexity and available computing resources.

As shown, the physiological constraints 206 contain one rule, which states that if the main-rhythm feature value is “2,” the secondary-rhythm feature must not have a value of “2.” The first row shows the possible main-rhythm values identified as possible, namely “1” and “2.” Other information may be included but is omitted for clarity, including confidence values, time stamps, log entries, etc.

Secondary-rhythm values then found, with possible values of “1,” “2,” and “3.” Of note, when the primary-rhythm values and secondary-rhythm values are aggregated, sets of possible combinations are created. Given that the physiological constraints 206 restrict the group “2,2,” this group is not included in the second row.

Atrial-enlargement features are found, with the possible values of “Yes,” and “No.” As shown, combinations with the previous feature sets are made, though some are omitted for clarity. Similarly, features of the various classes are combined, and those have also been omitted for clarity.

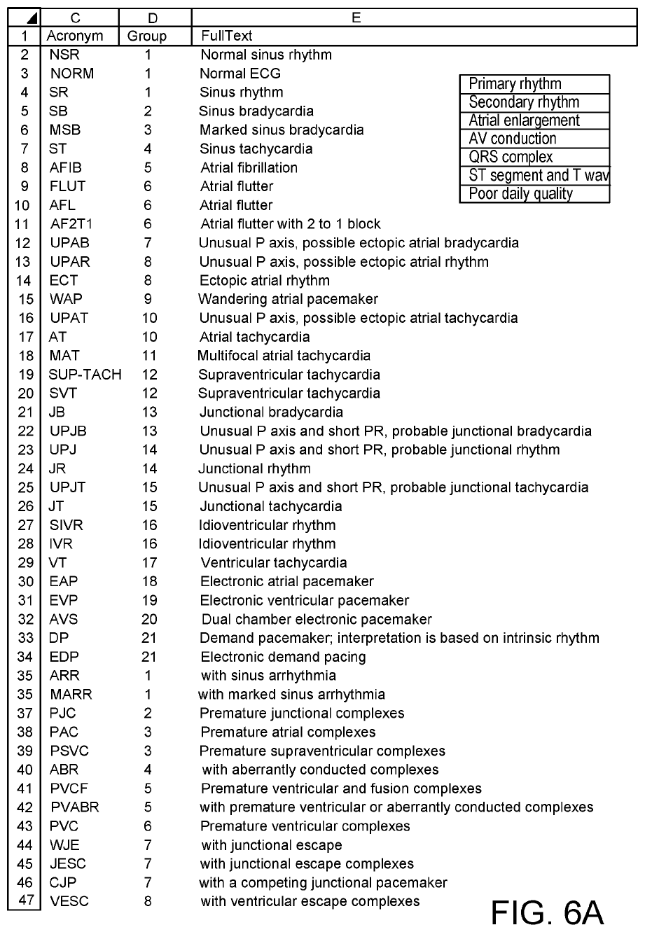

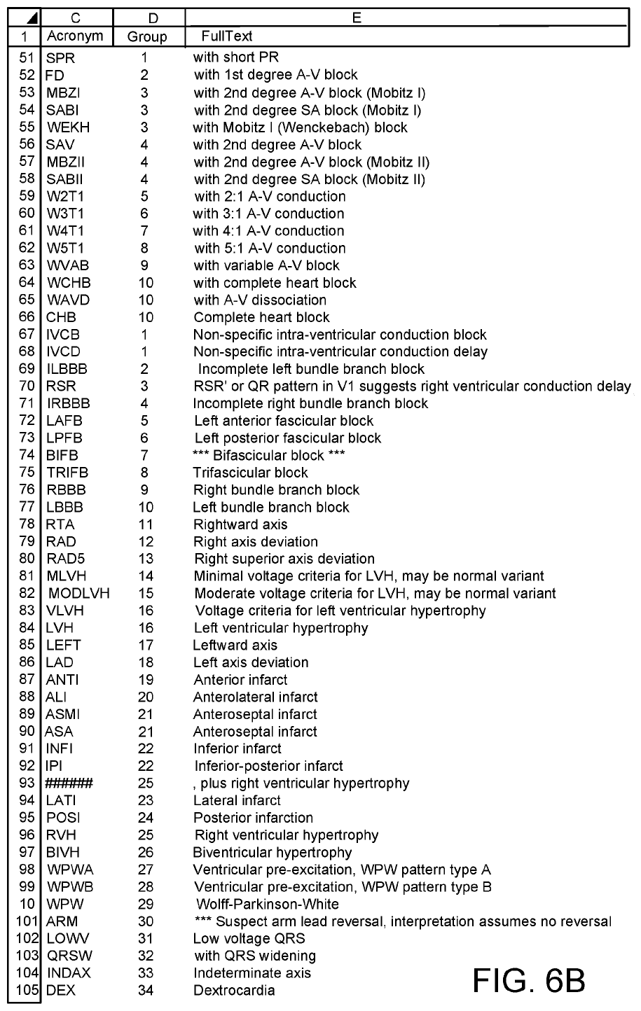

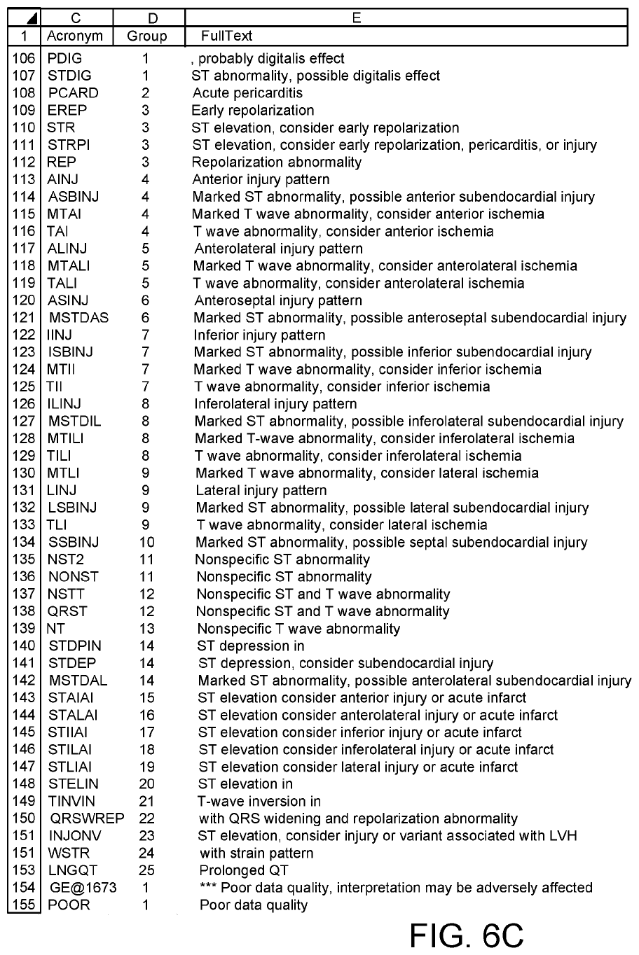

FIG. 6A-6B is a spreadsheet of example data that can be used in the processing of ECG data. This spreadsheet shows various cardiac features and presents one possible categorization and indexing scheme, though others are possible. As shown, group numbers may be used to aggregate similar features into a group number. Thus, in the previous example when a primary-rhythm value of “1” is used, that may indicate one of “Normal sinus rhythm,” “normal ECG,” or “sinus rhythm.”

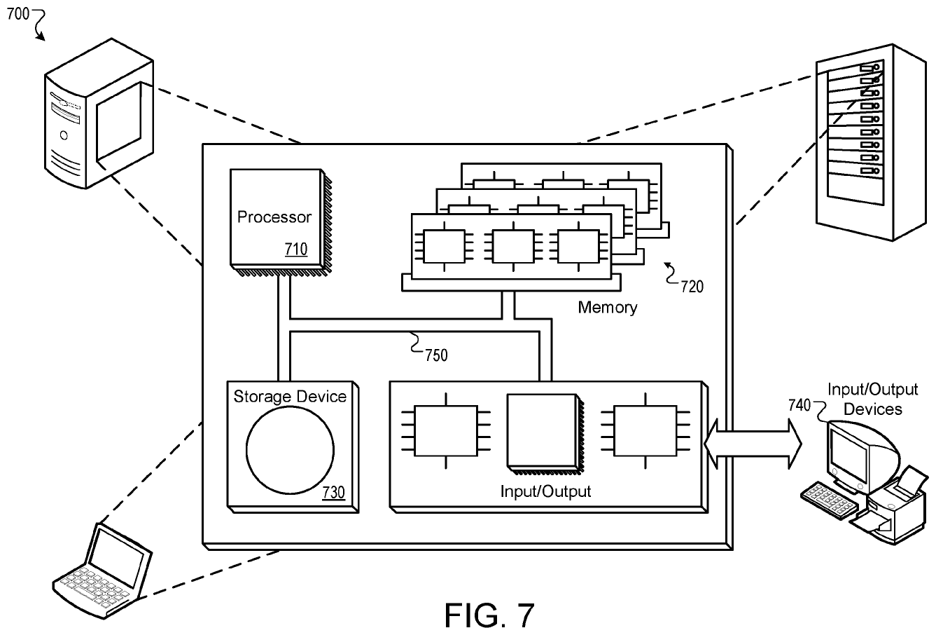

FIG. 7 is a schematic diagram that shows an example of a computing system 700. The computing system 700 can be used for some or all of the operations described previously, according to some implementations. The computing system 700 includes a processor 710, a memory 720, a storage device 730, and an input/output device 740. Each of the processor 710, the memory 720, the storage device 730, and the input/output device 740 are interconnected using a system bus 750. The processor 710 is capable of processing instructions for execution within the computing system 700. In some implementations, the processor 710 is a single-threaded processor. In some implementations, the processor 710 is a multi-threaded processor. The processor 710 is capable of processing instructions stored in the memory 720 or on the storage device 730 to display graphical information for a user interface on the input/output device 740. [56] The memory 720 stores information within the computing system 700. In some implementations, the memory 720 is a computer-readable medium. In some implementations, the memory 720 is a volatile memory unit. In some implementations, the memory 720 is a non-volatile memory unit.

The storage device 730 is capable of providing mass storage for the computing system 700. In some implementations, the storage device 730 is a computer-readable medium. In various different implementations, the storage device 730 may be a floppy disk device, a hard disk device, an optical disk device, or a tape device.

The input/output device 740 provides input/output operations for the computing system 700. In some implementations, the input/output device 740 includes a keyboard and/or pointing device. In some implementations, the input/output device 740 includes a display unit for displaying graphical user interfaces.

Some features described can be implemented in digital electronic circuitry, or in computer hardware, firmware, software, or in combinations of them. The apparatus can be implemented in a computer program product tangibly embodied in an information carrier, e.g., in a machine-readable storage device, for execution by a programmable processor; and method steps can be performed by a programmable processor executing a program of instructions to perform functions of the described implementations by operating on input data and generating output. The described features can be implemented advantageously in one or more computer programs that are executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, and the sole processor or one of multiple processors of any kind of computer. Generally, a processor will receive instructions and data from a read-only memory or a random access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM (erasable programmable read-only memory), EEPROM (electrically erasable programmable read-only memory), and flash memory devices; magnetic disks such as internal hard disks and removable disks; magneto-optical disks; and CD-ROM (compact disc read-only memory) and DVD-ROM (digital versatile disc read-only memory) disks. The processor and the memory can be supplemented by, or incorporated in, ASICs (application-specific integrated circuits).

To provide for interaction with a user, some features can be implemented on a computer having a display device such as a CRT (cathode ray tube) or LCD (liquid crystal display) monitor for displaying information to the user and a keyboard and a pointing device such as a mouse or a trackball by which the user can provide input to the computer.

Some features can be implemented in a computer system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network. Examples of communication networks include, e.g., a LAN (local area network), a WAN (wide area network), and the computers and networks forming the Internet.

The computer system can include clients and servers. A client and server are generally remote from each other and typically interact through a network, such as the described one. The relationship of client and server arises by virtue of computer programs running on the respective computers and having a client-server relationship to each other.

Although a number of implementations have been described in detail above, other modifications are possible. In addition, the logic flows depicted in the figures do not require the particular order shown, or sequential order, to achieve desirable results. Other steps may be provided, or steps may be eliminated, from the described flows, and other components may be added to, or removed from, the described systems. Accordingly, other implementations are within the scope of the following claims.

### EXAMPLE 1

Arrhythmia Detection using ECG

An arrhythmia is an abnormal heart rate and/or rhythm due to certain conditions with the heart electrical conducting system. Accurate detection of arrhythmia from ECG is a hard task that is mostly done by a trained cardiologist. Most current algorithms for arrhythmia detection have low accuracy and are trained using hand crafted features and predefined decision rules.

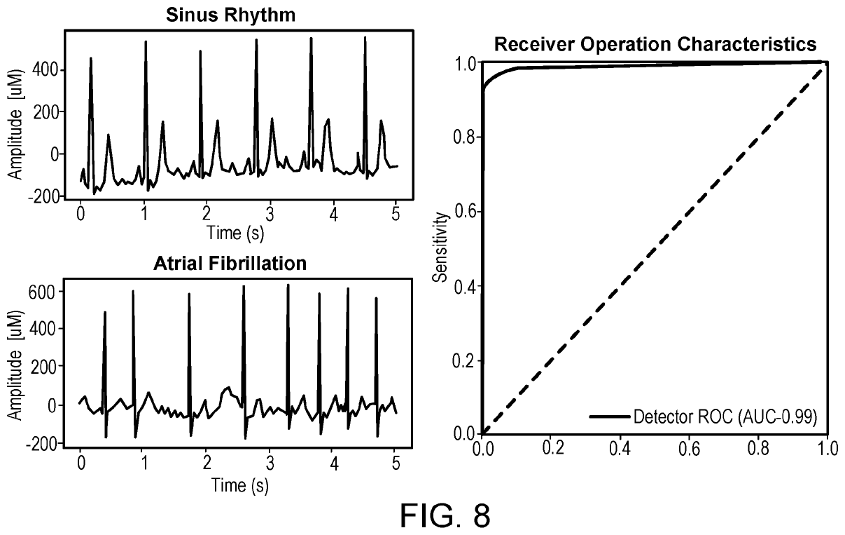

Referring to FIG. 8, atrial fibrillation (AFib) is an irregular and often rapid heart rate. At least 2.7 million Americans are living with AFib and if untreated, it is associated with a 5-fold increased risk for stroke. We trained a deep convolutional neural network to detect the presence of atrial fibrillation in a ten second single lead (Lead I) ECGs. A cohort of 97,829 patients was used to develop and test the algorithm. In the 52,870 patients tested, 4,911 had a verified atrial fibrillation rhythm during the time of ECG, 57% of patients were males and the mean age was 61.8 +−16.5. The Area Under the Curve (AUC) for both an internal validation set and the testing dataset was 0.99 and a test threshold was calculated using the development. The detector sensitivity was 0.97 with a specificity of 0.96 and an overall accuracy of 0.96

1. A system for identifying features of an electrocardiogram (ECG) of a subject, comprising:
at least one sensor contactable with the subject; and
an element adapted to receive signals from said sensor and configured to determine a plurality of features of an ECG generated from the signals by submission of the ECG to a plurality of machine-learning classifiers that are each configured to identify some, but not all, features out of a complete feature-set.

2. A system for generating data that describes features of an ECG of a subject, the system comprising:
one or more hardware processors; and
non-transitory computer-readable memory containing instructions that, when processed by the processor, cause the system to perform operations comprising:
receiving ECG data that was generated to reflect cardiac activity of a particular mammal;
submitting the ECG data to a plurality of cardiac classifiers, each cardiac classifier configured to identify, in the ECG, at least some of a plurality of cardiac features that are within a particular feature-class;
receiving, from each of the plurality of cardiac classifiers, a classification message containing data of the cardiac classifiers identifying of cardiac features in the ECG; and
assembling, from the received classification messages, ECG features for the ECG, the ECG features identifying at least some features of different feature-classes.

3. The system of claim 2, wherein the plurality of cardiac classifiers comprises:
a main-rhythm-classifier configured to identify, in the ECG, at least some main-rhythm features;
a secondary-rhythm-classifier configured to identify, in the ECG, at least some secondary-rhythm features;
an atrial-enlargement-classifier configured to identify, in the ECG, at least some atrial-enlargement features;
an atrioventricular (AV)-conduction main-rhythm-classifier configured to identify, in the ECG, at least some AV-conduction features;
a QRS-classifier configured to identify, in the ECG, at least some QRS-features;
a ST\T-wave-classifier configured to identify, in the ECG, at least some ST\T-wave features; and
a noisy-classifier configured to identify, in the ECG, at least some noisy features.

4. The system of claim 2, wherein at least some of the cardiac classifiers are configured to access physiological-constraint data that defines constraints on possible feature identification such that identified features are constrained to only features that are physiologically possible in a single given ECG.

5. The system of claim 2, wherein at least one of the cardiac classifiers are configured to identify three or more cardiac features that are within a particular feature-class.

6. The system of claim 2, wherein a second plurality of cardiac classifiers are at least some of the cardiac classifiers that are each configured to identify cardiac features within a particular feature-class; and
wherein the second plurality of cardiac classifiers are arranged in a decision tree such that identification of some cardiac features by a first cardiac classifier of the second plurality of cardiac classifiers causes a second cardiac classifier to identify at least one cardiac feature within the particular feature-class.

7. A non-transitory computer-readable memory comprising instructions that, when executed by one or more processors, cause the processors to perform operations for generating data that describes features of an ECG of a subject, the operations comprising:
receiving ECG data that was generated to reflect cardiac activity of a particular mammal;
submitting the ECG data to a plurality of cardiac classifiers, each cardiac classifier configured to identify, in the ECG, at least some of a plurality of cardiac features that are within a particular feature-class;
receiving, from each of the plurality of cardiac classifiers, a classification message containing data of the cardiac classifiers identifying of cardiac features in the ECG; and
assembling, from the received classification messages, ECG features for the ECG, the ECG features identifying at least some features of different feature-classes.

8. The non-transitory computer-readable memory of claim 7, wherein the plurality of cardiac classifiers comprises:
a main-rhythm-classifier configured to identify, in the ECG, at least some main-rhythm features;
a secondary-rhythm-classifier configured to identify, in the ECG, at least some secondary-rhythm features;
an atrial-enlargement-classifier configured to identify, in the ECG, at least some atrial-enlargement features;
an atrioventricular (AV)-conduction main-rhythm-classifier configured to identify, in the ECG, at least some AV-conduction features;
a QRS-classifier configured to identify, in the ECG, at least some QRS-features;
a ST\T-wave-classifier configured to identify, in the ECG, at least some ST\T-wave features; and
a noisy-classifier configured to identify, in the ECG, at least some noisy features.

9. The non-transitory computer-readable memory of claim 7, wherein at least some of the cardiac classifiers are configured to access physiological-constraint data that defines constraints on possible feature identification such that identified features are constrained to only features that are physiologically possible in a single given ECG.

10. The non-transitory computer-readable memory of claim 7, wherein at least one of the cardiac classifiers are configured to identify three or more cardiac features that are within a particular feature-class.

11. The non-transitory computer-readable memory of claim 7, wherein a second plurality of cardiac classifiers are at least some of the cardiac classifiers that are each configured to identify cardiac features within a particular feature-class; and
wherein the second plurality of cardiac classifiers are arranged in a decision tree such that identification of some cardiac features by a first cardiac classifier of the second plurality of cardiac classifiers causes a second cardiac classifier to identify at least one cardiac feature within the particular feature-class.

12. The non-transitory computer-readable memory of claim 7, wherein a second plurality of cardiac classifiers are at least some of the cardiac classifiers that are each configured to identify cardiac features within a particular feature-class; and
wherein the second plurality of cardiac classifiers are arranged in a decision tree such that identification of some cardiac features by a first cardiac classifier of the second plurality of cardiac classifiers causes a second cardiac classifier to identify at least one cardiac feature within the particular feature-class.

13. A method for generating data that describes features of an ECG of a subject, the method comprising:
receiving ECG data that was generated to reflect cardiac activity of a particular mammal;
submitting the ECG data to a plurality of cardiac classifiers, each cardiac classifier configured to identify, in the ECG, at least some of a plurality of cardiac features that are within a particular feature-class;
receiving, from each of the plurality of cardiac classifiers, a classification message containing data of the cardiac classifiers identifying of cardiac features in the ECG; and
assembling, from the received classification messages, ECG features for the ECG, the ECG features identifying at least some features of different feature-classes.

14. The method of claim 13, wherein the plurality of cardiac classifiers comprises:
a main-rhythm-classifier configured to identify, in the ECG, at least some main-rhythm features;
a secondary-rhythm-classifier configured to identify, in the ECG, at least some secondary-rhythm features;
an atrial-enlargement-classifier configured to identify, in the ECG, at least some atrial-enlargement features;
an atrioventricular (AV)-conduction main-rhythm-classifier configured to identify, in the ECG, at least some AV-conduction features;
a QRS-classifier configured to identify, in the ECG, at least some QRS-features;
a ST\T-wave-classifier configured to identify, in the ECG, at least some ST\T-wave features; and
a noisy-classifier configured to identify, in the ECG, at least some noisy features.

15. The method of any of claim 13, wherein at least some of the cardiac classifiers are configured to access physiological-constraint data that defines constraints on possible feature identification such that identified features are constrained to only features that are physiologically possible in a single given ECG.

16. The method of any of claim 13, wherein at least one of the cardiac classifiers are configured to identify three or more cardiac features that are within a particular feature-class.

17. The method of any of claim 13, wherein a second plurality of cardiac classifiers are at least some of the cardiac classifiers that are each configured to identify cardiac features within a particular feature-class; and
wherein the second plurality of cardiac classifiers are arranged in a decision tree such that identification of some cardiac features by a first cardiac classifier of the second plurality of cardiac classifiers causes a second cardiac classifier to identify at least one cardiac feature within the particular feature-class.
